(19) United States
(12) Patent Application Publication
(10) Pub. No.: US 2016/0244807 A1
(43) Pub. Date: Aug. 25, 2016

(54) CHIP FOR BIOLOGICAL ANALYSES PROVIDED WITH WELLS HAVING AN IMPROVED SHAPE, CARTRIDGE INCLUDING THE CHIP AND METHOD FOR MANUFACTURING THE CHIP

(71) Applicant: Distretto Tecnologico Sicilia Micro e Nano Sistemi S.c.a.r.l., Catania (IT)

(72) Inventors: Sabrina Conoci, Tremestieri Etneo (IT); Maria Eloisa Castagna, Catania (IT); Andrea Lazzara, Pedara (IT); Roberta Giuffrida, Catania (IT)

(21) Appl. No.: 14/989,711

(22) Filed: Jan. 6, 2016

(30) Foreign Application Priority Data
Feb. 25, 2015 (IT) TO2015A000135

(51) Int. Cl. C12Q 1/68; B01L 7/00; B81C 1/00; B01L 3/00

(52) U.S. Cl. CPC C12Q 1/686; B01L 3/50851; B01L 7/52; C12Q 1/6813; B81C 1/00595; B01L 2300/0829; B01L 2300/18; B01L 2300/0861; B01L 2300/12; B01L 2200/12

(57) ABSTRACT

A chip for biological and/or biochemical analyses includes: a supporting body, including a substrate of semiconductor material and a structural layer of biocompatible material; and a plurality of wells, designed to contain a liquid solution for the biological and/or biochemical analyses. Each well is formed by: a bottom chamber, defining a first containment volume for containing the liquid solution, and a top chamber, extending at least partially vertically aligned to, and in fluid communication with, the bottom chamber and defining a second containment volume greater than the first containment volume. The bottom chamber is formed, at least in part, in the biocompatible structural layer.

### BACKGROUND

1. Technical Field

The present disclosure relates to a chip for biological/biochemical analyses provided with wells having an improved shape and to a method for manufacturing the chip. The present disclosure also regards a cartridge for biological/biochemical analyses that includes the chip.

2. Description of the Related Art

As is known, analysis of nucleic acids requires, according to various modalities of recognition and preliminary steps of preparation of a sample of biological material, of amplification of the nucleic material contained therein, and of detection of individual target or reference strands, corresponding to the sequences sought.

At the end of the preparatory steps, the sample typically is examined to check whether amplification has taken place regularly.

According to the methodology referred to as “real-time PCR”, DNA is amplified through appropriately selected thermal cycles, and evolution of amplification is detected and monitored by fluorescence throughout the process.

For this purpose, various inspection methods and apparatuses of an optical type are known. In particular, the methods and apparatuses of an optical type are frequently based upon the phenomenon of fluorescence. The amplification reactions are conducted in such a way that the strands, contained in a recognition chamber obtained in a substrate, include fluorescent molecules or fluorophores. The substrate is exposed to a light source having an appropriate spectrum of emission, such as to excite the fluorophores. In turn, the fluorophores, once excited, emit secondary radiation at an emission wavelength higher than the peak of the excitation spectrum. The light emitted by the fluorophores is collected and detected by an optical sensor.

Known chip-based systems present some limitations. In particular, the geometry and composition of the surface of the chip typically used for the analysis are the cause of undesired reflections of the optical source used for illuminating the substrate during fluorescence analysis, generating disturbance, which raises the noise threshold and thus lowers the detection sensitivity.

Furthermore, in systems of a known type, the containment of drops to be analyzed is performed using a surface-modification layer with hydrophobic characteristics, which surrounds a hydrophilic region of silicon. A material typically used as surface-modification layer is known under the trade name “SINK” and is manufactured by Shin-Etsu MicroSi. During the fluorescence analysis, however, there has been noted an undesired interference between the SINR pattern and the fluorescence emitted by the solution being analyzed, with a localized, and undesirable, increase of the fluorescence emitted.

There is thus felt the need to overcome the drawbacks of the known art.

### BRIEF SUMMARY

One embodiment of the present disclosure is to provide a chip for biological/biochemical analyses provided with wells having an improved shape, a cartridge for biological/biochemical analyses including said chip, and a method for manufacturing the chip that will enable the limitations of the known art to be overcome, and in particular will enable improvement of the mechanical confinement of the solution to be analyzed and increase the intensity of light radiation emitted.

According to the present disclosure, a chip for biological/biochemical analyses, a cartridge for biological/biochemical analyses including said chip, and a method for manufacturing the chip are provided.

### BRIEF DESCRIPTION OF THE SEVERAL VIEWS OF THE DRAWINGS

For a better understanding of the disclosure, some embodiments thereof will now be described purely by way of non-limiting example and with reference to the attached drawings, wherein:

FIG. 1 is a perspective view of a chip for biological analyses provided with wells with dual liquid-retention chamber, according to one aspect of the present disclosure;

FIG. 2 is a front view of the chip of FIG. 1;

FIG. 3 is a cross-sectional view taken along the line of section II-II of FIG. 2 of the chip of FIGS. 1 and 2;

FIGS. 4A-4H show, in cross-sectional view, steps for manufacture of the chip of FIGS. 1-3 according to one embodiment of the present disclosure;

FIGS. 5A-5D show, in cross-sectional view, steps of manufacture of a chip according to an embodiment alternative to that of FIGS. 4A-4H;

FIG. 6 shows a rear view of the chip of FIG. 1 or FIG. 5D, including a heater and a temperature sensor, which forms a cartridge for biological and/or biochemical analyses according to one aspect of the present disclosure; and

FIG. 7 illustrates steps of a method for loading the chip of FIG. 1 or FIG. 5D.

### DETAILED DESCRIPTION

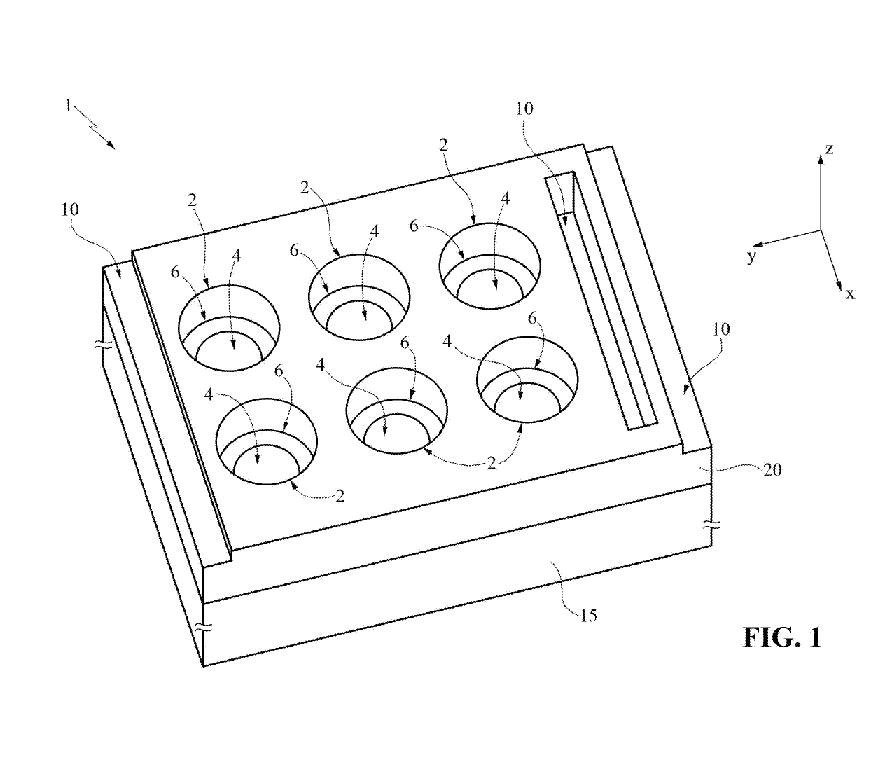

FIG. 1 shows, according to one aspect of the present disclosure, a chip 1 for biological and/or biochemical analyses, including a plurality of wells 2 (for example, six wells, but, twelve, or eighteen, or twenty-four wells, or any other number may be equally present, according to the need), which are designed to house a solution for biological and/or biochemical analyses. Each well 2 includes a bottom chamber 4, having a cylindrical shape, which has a diameter d₁, and a top chamber 6, which also has a cylindrical shape and has a diameter d₂ greater than the diameter d₁. For each well 2, the top chamber 6 extends as continuation of, and is in fluid connection with, the bottom chamber 4.

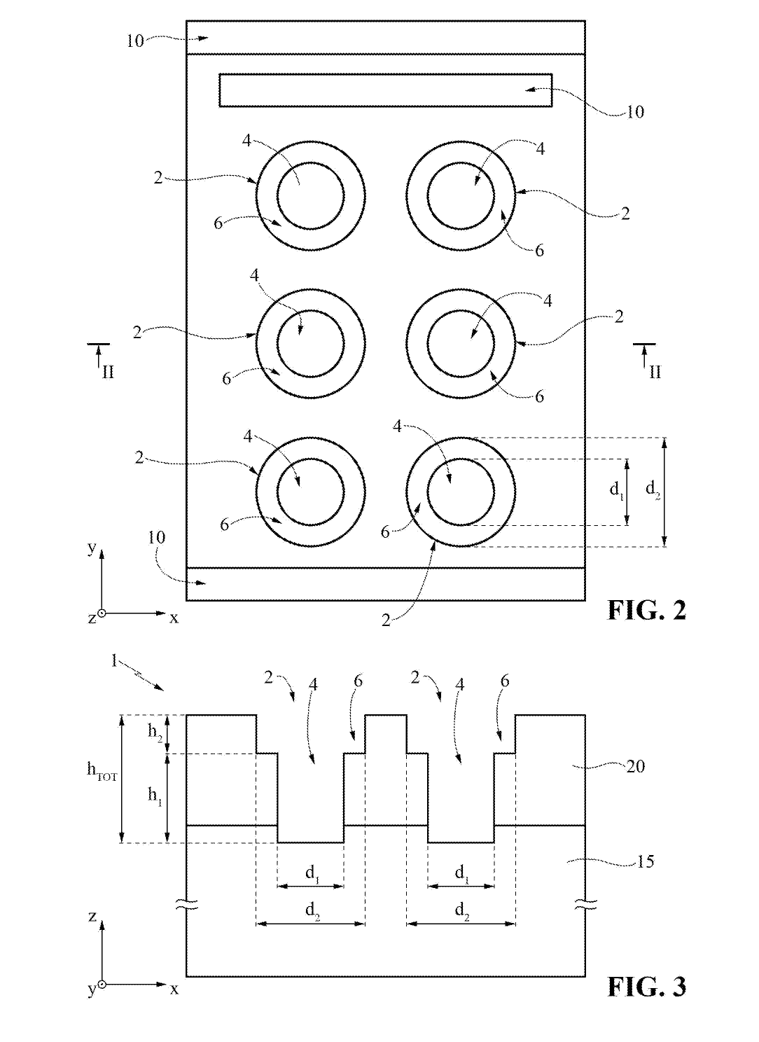

As may be noted from FIG. 2, the bottom chamber 4 and the top chamber 6 of each well have, in top view, a concentric circular shape, with respective diameters d₁ and d₂. In other words, the bottom chamber 4 and the top chamber 6, which are cylindrical, extend along a same axis of symmetry passing through the centroid of both of the cylinders, i.e., the axis Z. In particular, the chip 1 of FIGS. 1 and 2 is provided with six wells 2 of the type described, in which the diameter d₁ of the bottom chamber 4 is smaller than the diameter d₂ of the top chamber 6. For instance, the diameter d₁ is 2 mm, and the diameter d₂ is 3.3 mm.

According to the embodiment shown in FIGS. 1 and 2, the chip 1 has, in top plan view, a rectangular shape, with major side a having an extension, along an axis Y, of for example 17.6 mm and minor side b having an extension, along an axis X orthogonal to the axis Y, of for example 12.1 mm. The thickness c of the chip 1, i.e., its extension along an axis Z orthogonal to the axes X and Y, is for example 3 mm.

In greater detail, the wells 2 are arranged along two rows arranged alongside one another (three wells for each row) and the wells 2 belonging to a same row are aligned to one another along a same direction parallel to the axis Y. The wells 2 belonging to a same row are further separated from one another (along Y) by a minimum distance of, for example, 1.3 mm. Likewise, the wells 2 belonging to different rows are separated, along X, from a respective well 2 by a minimum distance of, for example, 1.3 mm. The distance between directly facing wells, along X or along Y, is measured on the outer edge of the respective top chamber 6.

It is evident that the dimensions indicated above are provided by way of example of a possible embodiment and may vary according to the need. In particular, in the case of a chip provided with twenty-four wells, it is advisable to maintain the dimensions a, b, and c coinciding with those of the chip 1 described above (for compatibility with the optical reading system) and reduce the dimensions of the bottom chamber 4 and top chamber 6 of each well 2, as likewise their mutual distance along X and along Y. For instance, the bottom chamber 4 of each well 2 may have a diameter d₁ of 1 mm, and the top chamber 6 of each well 2 may have diameter d₂ of 1.8 mm.

In general, the diameter d₁ is chosen between 0.5 and 4 mm, and the diameter d₂ is chosen between 1 and 6 mm (in any case satisfying the relation d₂>d₁).

FIG. 3 shows a cross-sectional view of the chip 1, taken along the line of section II-II of FIG. 2. Each well 2 has a total depth h_{TOT}, measured along the axis Z, chosen between 2.5 mm and 3.5 mm, for example, equal to 3 mm. The total depth h_{TOT} is given by the sum of the partial depths h₁ of the bottom chamber 4 and h₂ and of the top chamber 6. The bottom chamber 4 of each well has a depth h₁, along Z, chosen between 2 mm and 3.5 mm, while the top chamber 6 of each well has a depth h₂, along Z, chosen between 1 mm and 1.5 mm. However, the ratio between the depths identified here may vary as a function of the volumes required by the specific application. In particular, given the values of diameter d₁ and d₂, the depths h₁ and h₂ are chosen of respective values such as to satisfy requirements of minimum/maximum amount of liquid that each well 2 may contain. For instance, the dimensions of the bottom chamber 4 of each well 2 of the chip 1 of FIGS. 1-3 are such that each bottom chamber 4 may contain approximately 8-15 μl of a liquid solution. With reference to a same chip 1, the dimensions of the top chamber 6 are such that each top chamber 6 may contain approximately 19-12 μl of a liquid solution.

Once again by way of example, the dimensions of the bottom chamber of the wells of a chip with twelve wells are chosen with values such that each bottom chamber may contain approximately 4-6 μl of a liquid solution, whereas the dimensions of the top chamber 6 are such that each top chamber 6 may contain approximately 10-8 μl of a liquid solution.

Once again by way of example, the dimensions of the bottom chamber of the wells of a chip with twenty-four wells are chosen with values such that each bottom chamber may contain approximately 1-2 μl of a liquid solution, whereas the dimensions of the top chamber 6 are such that each top chamber 6 may contain approximately 3.5-2.5 μl of a liquid solution.

In general, each bottom chamber 4 is configured to contain a liquid solution in an amount comprised between 1 μl and 15 μl (i.e., each bottom chamber 4 defines a containment volume comprised between 1.2 mm³ and 10 mm³). Each top chamber 6 is to contain a liquid solution in an amount comprised between 3.8 μl and 19 μl (i.e., each top chamber 6 defines a containment volume comprised between 3 mm³ and 19 mm³).

According to one embodiment, the bottom chamber 4 of each well 2 is designed to contain a biological solution, whereas the top chamber 6, of greater volume, is designed to contain oil or wax having the function of preventing, during use and during the thermal cycles, evaporation of the underlying biological solution.

According to one aspect of the present disclosure, the chip 1 optionally includes one or more trenches 10, having the function of lightening the structure of the chip 1 and decreasing its thermal inertia.

According to the embodiment of FIGS. 1-3, the chip 1 comprises a supporting body 15, for example a substrate of semiconductor material such as silicon, and a biocompatible structural layer 20, made, for example, of polycarbonate, laid on top of the supporting body 15 (and fixed thereto by a fixing layer, for example biocompatible glue). The wells 2 are formed in part in the biocompatible structural layer 20 and in part in the supporting body 15. In particular, only the bottom chamber 4 is formed in part in the biocompatible structural layer 20 and in part in the supporting body 15. Still more in particular, the portion of the bottom chamber 4 that extends exclusively in the supporting body 15 has a depth (along Z) equal to, or smaller than, half of the total depth h₁ of the bottom chamber 4.

This embodiment presents the advantage of increasing the volume of the part of the chip 1 containing the biological solution and at the same time decreasing its thermal inertia, exploiting the thermal characteristics of a semiconductor substrate (for example, silicon, in particular silicon with N doping). The portion of each bottom chamber 4 that extends in the semiconductor substrate is comprised between some tens of micrometers and some hundreds of micrometers, for example between 50 and 700 μm, in particular 500 μm.

The portion of bottom chamber 4 formed in the supporting body 15 has the same diameter d₁ as the portion of bottom chamber 4 formed in the biocompatible structural layer (but for the tolerances due to the process of formation of the holes that provide the bottom chamber 4). The portion of the wells 2 formed in the semiconductor substrate is dug by lithographic and etching steps.

Thus, according to the embodiment of FIGS. 1-3, at the end of the manufacturing process, the chambers containing the biological solution are formed in part by the volume dug in the semiconductor substrate (silicon) and in part by the volume provided in the biocompatible layer (polycarbonate).

According to a different embodiment, the wells 2 are formed exclusively in the biocompatible structural layer, made, for example, of polycarbonate. Said structural layer is in turn coupled (e.g., by gluing) to a smooth surface of a supporting body of semiconductor material, e.g., silicon, which forms the bottom of the wells 2 (i.e., the bottom of the bottom chambers 4 of the wells 2). The semiconductor body may integrate, as illustrated more fully hereinafter, heaters and/or temperature sensors.

Described in greater detail in what follows are methods for manufacturing the chip 1, according to the respective embodiments.

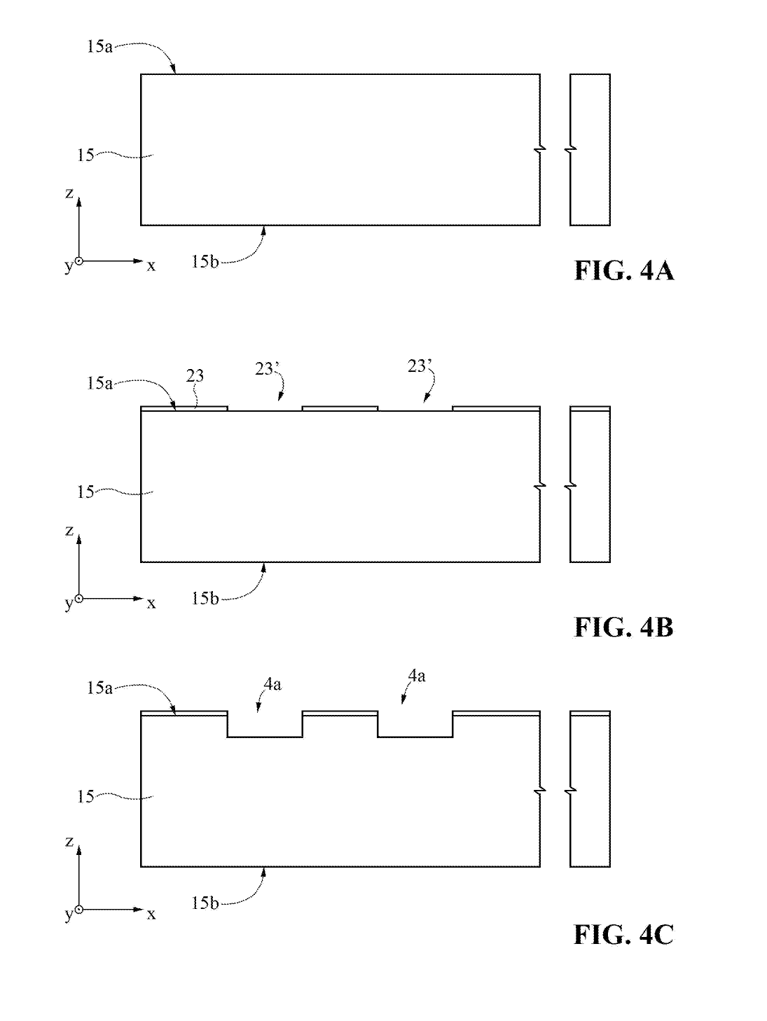

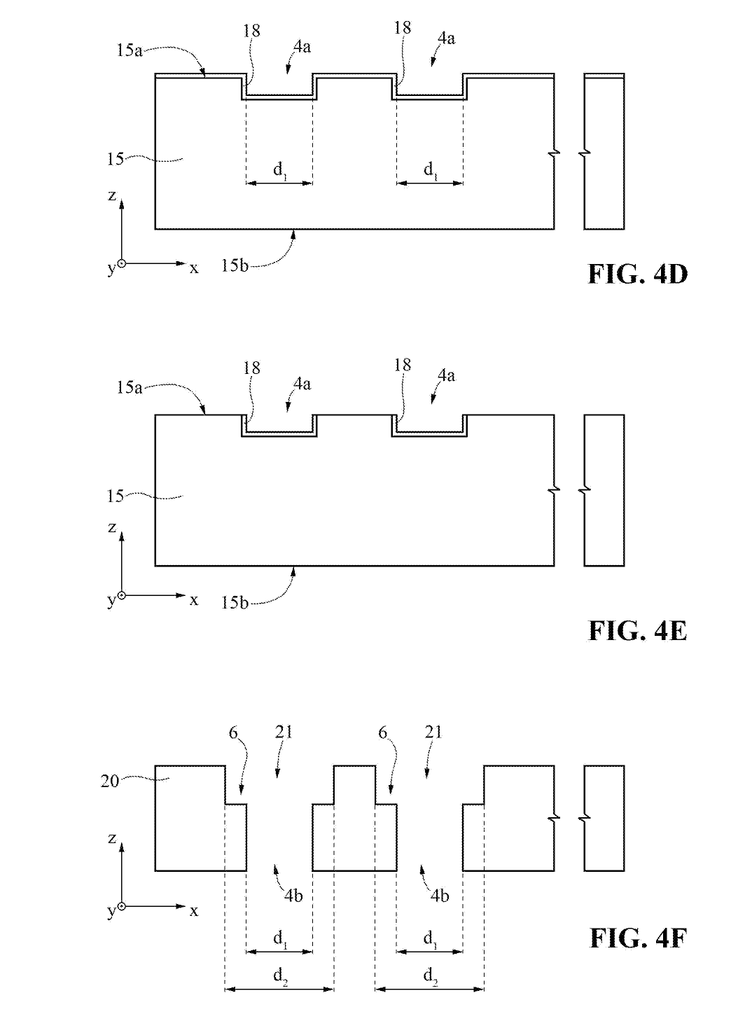

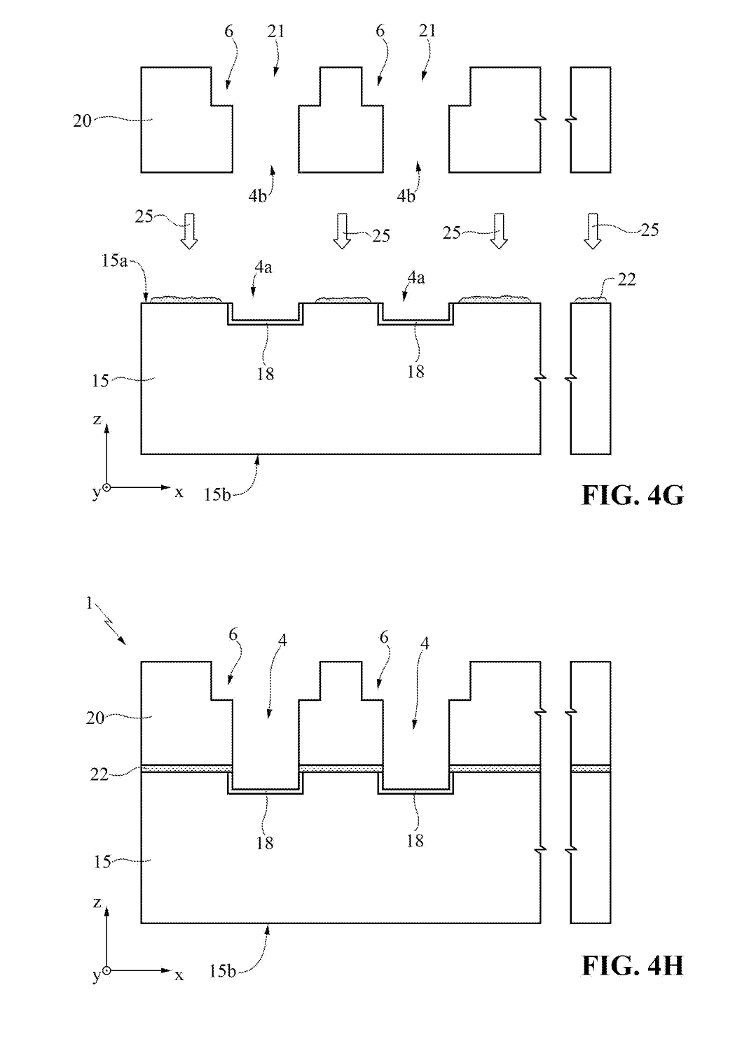

With reference to FIGS. 4A-4H a method for manufacturing the wells 2 of the chip 1 is now described.

With reference to FIG. 4A, a supporting body 15, or substrate, is provided, of semiconductor material (e.g., silicon), having a front side 15a and a rear side 15b, opposite to one another along the axis Z. Said supporting body 15 may include a wafer of semiconductor material, possibly previously processed. In particular, the semiconductor wafer may be doped with dopant species of an N type.

Next, a first portion 4a of the bottom chambers 4 is formed, by successive steps of masking and lithography (FIG. 4B) in order to define, at the front 15a, the regions in which the first portion 4a of the bottom chambers 4 are formed. Then a chemical etch (FIG. 4C), for example of a dry type, in particular a plasma etch, removes selective portions of the supporting body 15, to form the first portion 4a of the bottom chambers 4.

In particular, with reference to FIG. 4B, a mask 23 of photoresist or silicon oxide (SiO₂) is formed, which defines openings 23′ through which portions of the front 15a of the supporting body 15 are exposed, said portions having, in top plan view, a circular shape, and dimensions (diameter d₁) such as to enable formation of the bottom chambers 4.

With reference to FIG. 4D, the mask 23 is removed, and a step of thermal growth of oxide (Si O₂) is carried out to form a biocompatible layer 18 that uniformly covers the bottom and the internal wall of each first portion 4a of the respective bottom chamber 4. Each first portion 4a thus formed has a depth of, for example, 500 μm.

It is evident that the step of FIG. 4D of growth of the biocompatible layer 18 entails formation of thermal oxide also on the outside of the bottom chambers 4, i.e., on the front 15a of the substrate 15.

Alternatively, it is possible to carry out the etching step of FIG. 4C and the step of thermal growth of FIG. 4D prior to removal of the mask layer 23. In this way, as shown in FIG. 4E, after thermal growth of the biocompatible layer 18 selectively inside the bottom chambers 4, the mask 23 is removed, and there is no formation of thermal oxide on the front 15a of the substrate 15.

With reference to FIG. 4F, a second portion 4b of the bottom chambers 4 and of the top chambers 6 is formed in a supporting body 20 of biocompatible material, in particular polycarbonate or PDMS.

In the case of a supporting body 20 of polycarbonate, the supporting body 20 is provided, and formed therein is a plurality of through openings 21 (in particular, of a cylindrical shape), having the shape previously described for the wells 2, i.e., each having a bottom through opening, with a diameter d₁, fluidically connected to a top through opening, having a diameter d₂>d₁. The bottom through opening provides the second portion 4b of the respective bottom chamber 4, whereas the top through opening provides the respective top chamber 6 of the respective well 2.

The number of through openings 21 is equal to the number of first portions 4a of the bottom chambers 4 formed in the substrate 15, i.e., equal to the number of wells 2 that are to be formed in the chip 1. Each through opening 21 is formed, for example, by an injection-molding technique or by mechanical milling, according to the material used to provide the supporting body 20.

Then (FIG. 4G), a step of coupling of the substrate 15 with the supporting body 20 is carried out. For this purpose, a coupling layer 22, for example epoxy glue, or silicone, or some other biocompatible glue, is formed at the front 15a of the substrate 15, in regions of the latter comprised between the first portions 4a of the bottom chambers 4. Alternatively, or in addition, a coupling layer of glue or silicone, is formed in surface regions of the supporting body 20.

Next, a step of mechanical coupling between the substrate 15 and the supporting body 20 is carried out (represented schematically by the arrows 25 of FIG. 4G), which are arranged in contact with one another in such a way that the centroid of each first portion 4a of the bottom chambers 4 is aligned, along the axis Z, with a respective centroid of a respective second portion 4b formed in the supporting body 20.

There is thus formed (FIG. 4H) a chip 1 according to one aspect of the present disclosure, in which the through openings 21 provide the top chambers 6 and, at least in part, the bottom chambers 4 of each well 2.

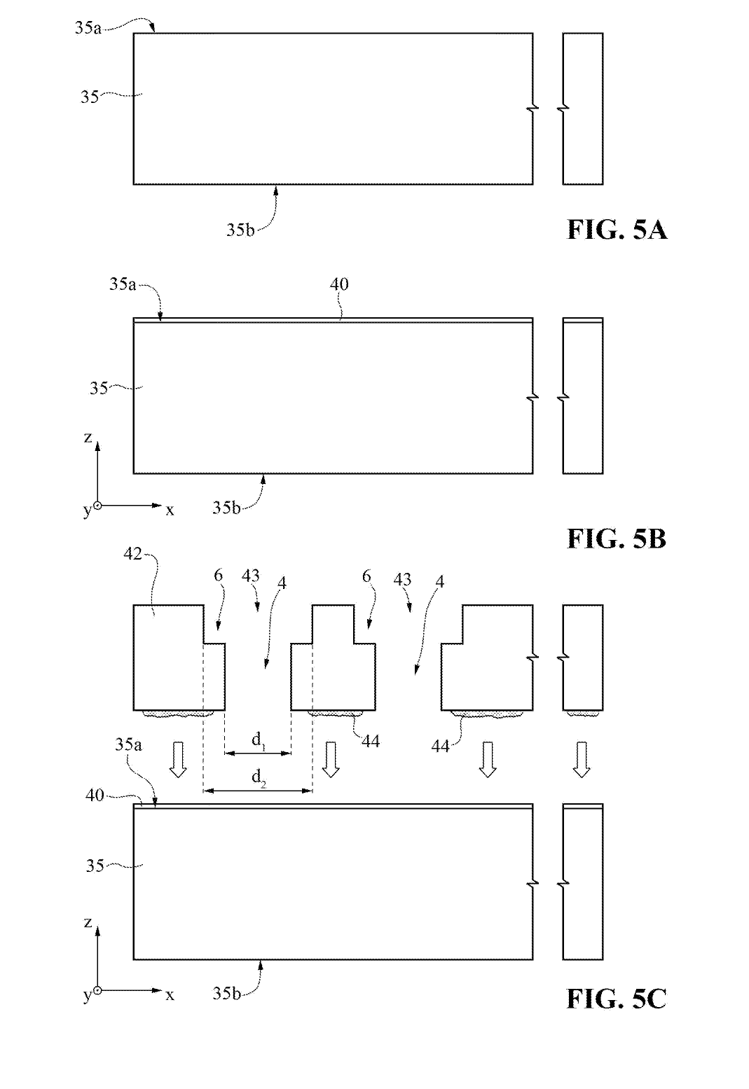

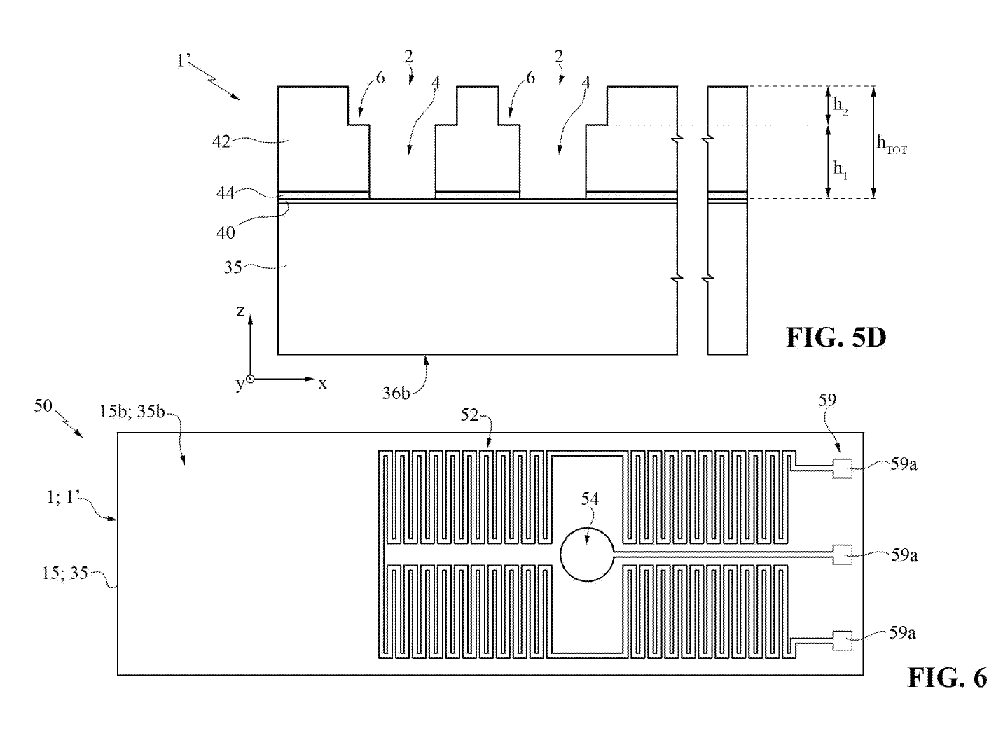

FIGS. 5A-5D show steps for manufacture of a chip 1 according to a further embodiment of the present disclosure. In particular, FIGS. 5A-5D describe manufacturing steps in which the top chambers 6 and bottom chambers 4 of the wells 2 are formed completely in the supporting body 20, and a semiconductor substrate has the function of bottom wall of said wells 2.

With reference to FIG. 5A, a supporting body, or substrate, 35 is provided of semiconductor material (e.g., silicon), having a front side 35a and a rear side 35b, opposite to one another along the axis Z. Said supporting body 35 may be of a previously processed type (for example, it may comprise one or more epitaxial layers, for instance having the purpose of increasing the thickness of the starting wafer for strengthening the chip 1).

Optionally (FIG. 5B), it is possible, starting from the supporting body 35 of FIG. 5A, to carry out a step of thermal growth of silicon oxide for forming a biocompatible layer 40 on the front side 35a.

Then (FIG. 5C), the bottom chambers 4 and the top chambers 6 are formed in a structural layer 42 of biocompatible material, in particular polycarbonate or PDMS.

In the case of a polycarbonate structural layer 42, the structural layer 42 is provided, and formed therein is a plurality of through openings 43 (in particular, of a cylindrical shape), having the shape previously described for the wells 2, i.e., having each a bottom opening, with a diameter d₁ and a depth h₁, fluidically connected to a top opening, with a diameter d₂>d₁ and a depth h₂. Each bottom opening provides a respective bottom chamber 4, and each top opening provides a respective top chamber 6 of the respective well 2.

Each through opening 43 is formed, for example, by a technique of injection molding, or by mechanical milling according to the material used to obtain the structural layer 42.

Next, a step of coupling of the substrate 35 with the structural layer 42 is carried out. For this purpose, a coupling layer 44, made for example of epoxy glue, or silicone, or some other biocompatible glue, is formed in surface regions of the structural layer 42 that will be arranged in contact with the front side 35a of the substrate 35.

As shown in FIG. 5D, mechanical coupling between the substrate 35 and the structural layer 42 is obtained by arranging in contact with one another the substrate 35 and the structural layer 42 through the layer of glue 44.

A chip 1′ according to a further aspect of the present disclosure is thus formed, in which both the bottom chambers 4 and the top chambers 6 of each well 2 are formed exclusively through the structural layer 42, whereas the substrate 35 forms the bottom of the bottom chambers 4.

According to further embodiments, the wells 2 obtained independently according to the steps of FIGS. 4A-4H or of FIGS. 5A-5D may be functionalized for improving biocompatibility, by fixing BSA (bovine-serum albumin) protein to the walls and/or to the bottom of the wells 2, in a per se known manner. Functionalization may be carried out at the end of the steps for manufacture of the respective chip, or else during intermediate manufacturing steps.

In particular, according to one embodiment, the wells 2 are treated in order to improve biocompatibility thereof.

The treatment comprises a step of cleaning and activation, and includes a treatment with a solution of CH₃OH:HCl (4:1) for 10 minutes at room temperature, and, next, a step of rinsing with ultrapure water with pH of 7.0 to remove the excess reagents. There then follows a step of anhydration comprising a thermal treatment in oven for 15 minutes at 70° C.

These steps have the function of rendering the wells 2 (in particular, the bottom chamber 4 or the bottom of the bottom chamber 4) hydrophilic.

Then, the active surface of the wells 2 is further treated, during a blocking step, comprising a treatment with a solution including 1% BSA, 5% SSC (sodium chloride plus sodium citrate). This step is carried out, in particular, at 55° C. for a time comprised between 4 and 15 hours, in which the solution is left to rest in the wells 8.

Finally, washing with deionized water is carried out.

Since the treatment described previously for increasing hydrophilicity may inhibit PCR on account of the presence of polyelectrolytes, the latter steps have the function of restoring characteristics suitable for PCR so that it may take place correctly and as desired.

According to one aspect of the present disclosure (FIG. 6), irrespective of the manufacturing method adopted, the chip 1 may be provided with one heater or a number of heaters and one temperature sensor or a number of temperature sensors.

As illustrated in FIG. 6, the chip 1, and likewise the chip 1′, includes a heater 52 and a temperature sensor 54, formed integrated in the substrate 15, 35, thus providing a microreactor 50 for biochemical and/or biological analyses.

Alternatively, one or both between the heater 52 and the temperature sensor 54 may be external to the chip 1, 1′ and thermally coupled to the rear side 15b, 35b of the substrate 15, 35 during use and only when necessary.

The heater 52 includes a plurality of resistive coils designed to develop heat by the Joule effect when they are traversed by current. The coils of the heater 52 extend, for example, in regions of the rear side 15b, 35b of the substrate 15, 35 substantially corresponding to respective regions of the front side 15a, 35a of the substrate 15, 35 that house the wells 2. The heater 52 and the temperature sensor 54 are thermally coupled to the wells 2 (in particular, to the bottom chamber 4), in such a way that the thermal energy released by the heater 52 will cause heating of the biological material in the wells 2 (in particular, in the bottom chamber 4). The heater 52 is defined by one or more conductive paths, for example of metal or polysilicon. The temperature sensor 54 is, for example, of a thermoresistive type.

The substrate 15, 35 may further comprise contact pads 59a arranged at a longitudinal end of the rear side 15b, 35b of the substrate 15, 35 for forming a connector 59. The connector 59 extends outside the area of the front side 15a, 35a that houses the wells 2. The connector 59 is electrically coupled to the heater 52 and to the temperature sensor 54 by conductive paths provided in the substrate 15, 35. The connector 59 enables control of the chip 1, 1′ (e.g., to carry out the thermal PCR cycles) once the chip 1 has been introduced into an analyzer.

Fluorescence analysis of the PCRs that take place in said microreactor may be carried out by a real-time PCR analyzer of the type described in the above document US 2013/0004954.

In order to carry out analyses on a sample using the chip 1, 1′, a mixture of reagents in solution that comprises fluorophores of two types is introduced into the wells 2. For instance, a first type of fluorophores (e.g., FAM) has an excitation wavelength λ_{E1} and a detection (or emission) wavelength λ_{D1} and combines with a first substance to be sought. A second type of fluorophores (e.g., ROX) has an excitation wavelength λ_{E2} and a detection (or emission) wavelength λ_{D2} and combines with a second, control, substance. The second type of fluorophores has the sole function of control marker, whereas the function of molecular probe for detecting DNA amplification is guaranteed by the fluorophores of the first type.

The light emitted by a source arranged at the top mouth of the wells 2 tends to follow the entire path present within the wells 2 (depth h₁+h₂) exciting in a uniform way the solution present in the wells themselves, considerably reducing the presence of shaded areas that would otherwise be present. Furthermore, having the solution in contact with the walls of the wells 2, which in particular are of polycarbonate, enables optimization of excitation of the fluorophores within the solution by the beams reflected by the polycarbonate walls. This enables an improvement in the excitation of the solution also in points usually in the shade such as, for example, the bottom vertex of the wells 2.

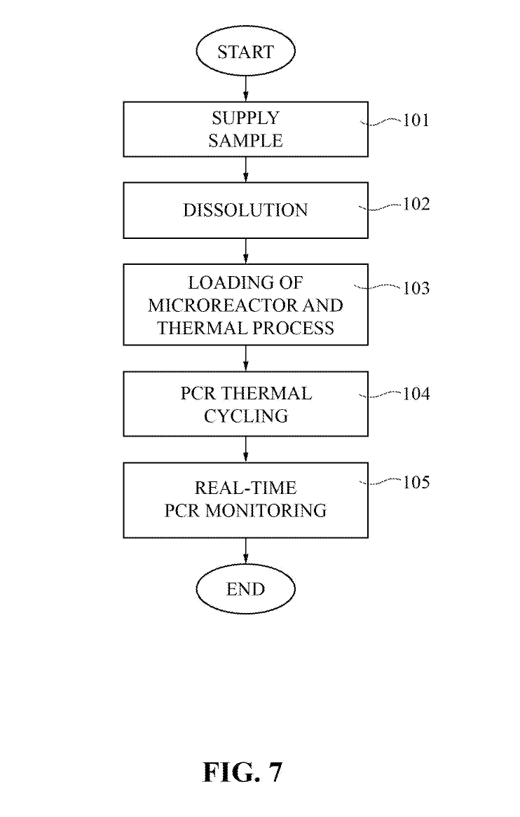

With reference to FIG. 7, according to a procedure of diagnosis of genetic diseases, a first step 101 is carried out in which a sample is provided, for example a sample of diagnostic buccal swab. The sample of buccal swab may be acquired by simple sampling of cells of the buccal epithelium, with a non-invasive procedure, then dispersing (step 102) said epithelial cells in a transport medium of a known type (dissolving step).

The dissolving step includes dissolution of the cells sampled in a transport medium (or transport solution). The transport solution is designed to favor lysis of the epithelial cells sampled for releasing the DNA. Lysis is completed during the initial steps of thermal cycling of the subsequent steps (favored by heat).

In use, to carry out the aforesaid analyses, each well 2 of the chip 1, 1′ is pre-loaded (step 103), with (the amounts indicated hereinafter refer by way of example to the chip 1, 1′ with six wells 2):
between 8 and 19 μl of wax (which is melted and poured in each well 2) and has the purpose of sealing each well 2 to prevent phenomena of evaporation during thermal PCR cycling;
between 0.5 and 8 μl of PCR reagents (including primers, probes, and polymerase enzyme), in the form of liquid solution; and
between 0.5 and 8 μl of sample solution containing nucleic acid (DNA/RNA) to be detected.

The wax used must be compatible with PCR and with the reagents used, in particular, it must not inhibit PCR. Furthermore, when melted, it must be transparent and exhibit a low fluorescence at the wavelengths of interest (used for fluorescence analysis) in order not to interfere with the measurements of the fluorescence emitted by the wells 2. Furthermore, it must preferably show a low vapor pressure in order not to evaporate during the thermal cycles required for PCR. Preferably, it must show a density lower than the density of the PCR reagents and the solutions used in such a way that it covers the PCR reagents and the solution in order to prevent evaporation thereof during the PCR thermal cycles. This further enables introduction into the wells 2 of PCR reagents/solution to be analyzed independently before or after introduction of the wax into the wells 2 (the wells 2 may thus be pre-loaded with the wax during the steps for manufacture of the chip 1, 1′). Furthermore, the wax affords an effective and valid protection of the wells 2, preventing phenomena of cross contamination between adjacent wells 2 and by external contaminating agents. In addition, the wax used must have an adequate melting point, such that it is solid at room temperature (or temperatures lower than room temperature), but liquid at the temperatures at which PCR is carried out (typically equal to or greater than approximately 55° C.).

The present applicant has found that a wax that possesses these characteristics is a paraffin wax, in particular of the type marketed by Sigma-Aldrich with code No. 76228.

As an alternative to wax, it is likewise possible to use mineral oil.

Then (step 104), the chip 1 thus loaded is inserted into the analyzer (e.g., into the analyzer described in US 2013/0004954), and PCR thermal cycling is started.

For this purpose, by way of example, the thermal cycling comprises heating to a temperature chosen in the range between 94 to 99° C. for a time comprised between 2 and 10 minutes, and then a plurality of cycles (e.g., 50 cycles), where each cycle includes:
a step of heating to a temperature comprised between 94 and 99° C. for a time comprised between 5 and 20 s; and
a step of heating to a temperature comprised between 57 and 62° C. for a time comprised between 35 and 70 s.

During these thermal cycles, the PCR process is monitored (step 105) by fluorescence analysis. However, any other real-time monitoring method, of a type known in the art, may be used (for example as described in US 2013/0004954).

From the foregoing disclosure, the advantages of the disclosure described are evident.

In the first place, the manufacturing method is simple and inexpensive, rendering the chip suited for applications of a single-use type.

Furthermore, the dual retention chamber for each well enables housing of the biological sample exclusively in the bottom chamber of the well directly in contact with the silicon, which ensures an efficient heat exchange, and housing of the sealing wax, or oil, exclusively in the top chamber of the well itself In this way, the present applicant has found that, as a result of the geometry, the sensitivity of detection of the fluorescence emitted increases as compared to diagnostic chips of a known type.

Further, since the bottom chambers of the wells are of silicon, heat exchange is optimized. In particular, optimization of heat exchange is noted when the silicon in which the bottom chambers are dug is doped.

Modifications and variations may be made to the device and method described herein, without thereby departing from the scope of the present disclosure. For instance, the chip 1, 1′ and the microreactor 50, 60 described may find application also in systems for carrying out biochemical processes other than PCR amplification and for recognition of the results thereof.

The various embodiments described above can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A chip for biological and/or biochemical analyses, comprising:
a supporting body including a substrate of semiconductor material and a structural layer of biocompatible material extending over the substrate; and
a plurality of wells which extend in the supporting body and are adapted to contain a liquid solution for said biological and/or biochemical analyses, each well including:
a bottom chamber defining a first containment volume for containing said liquid solution, and
a top chamber extending at least partially on, and in fluidic communication with, the bottom chamber and defining a second containment volume greater than the first containment volume,
wherein the top chamber extends completely in the structural layer, and the bottom chamber extends at least in part in the structural layer.

2. The chip according to claim 1, wherein both the bottom chamber and the top chamber extend completely in the structural layer and throughout a thickness of the structural layer.

3. The chip according to claim 2, wherein said structural layer is of a material having a refractive index lower than a refractive index of said liquid solution.

4. The chip according to claim 3, wherein the liquid solution is water-based, the substrate is of silicon, and the structural layer is of polycarbonate.

5. The chip according to claim 1, comprising a biocompatible adhesive layer that couples the structural layer to the substrate.

6. The chip according to claim 1, wherein, for each well:
the bottom chamber is cylindrical with a first base radius; and
the top chamber is cylindrical with a second base radius greater than the first base radius,
wherein the bottom chamber and the top chamber extend along a same axis of symmetry.

7. The chip according to claim 1, wherein the first containment volume of the bottom chamber is comprised between 1.2 mm³ and 10.6 mm³, and the second containment volume of the top chamber is comprised between 3.8 mm³ and 19 mm³.

8. A cartridge for biological and/or biochemical analyses comprising:
a chip that includes:
a supporting body including a substrate of semiconductor material and a structural layer of biocompatible material extending over the substrate; and
a plurality of wells which extend in the supporting body and are adapted to contain a liquid solution for said biological and/or biochemical analyses, each well including:
a bottom chamber defining a first containment volume for containing said liquid solution, and
a top chamber extending at least partially on, and in fluidic communication with, the bottom chamber and defining a second containment volume greater than the first containment volume, wherein the top chamber extends completely in the structural layer, and the bottom chamber extends at least in part in the structural layer; and
a heater thermally coupled to the plurality of wells of the chip via the substrate.

9. The cartridge according to claim 8, wherein:
at least one bottom chamber of a well of said plurality of wells houses nucleic-acid probes for hybridization to respective target nucleic acids, and a solution containing one or more from among: primers capable of binding to the target nucleic acids, nucleotides, enzymes for nucleic-acid extension, and nucleic acids, and
the top chamber houses mineral oil or wax for preventing evaporation of said solution.

10. The cartridge according to claim 8, wherein said structural layer is of a material having a refractive index lower than a refractive index of said liquid solution.

11. The cartridge according to claim 8, comprising a biocompatible adhesive layer that couples the structural layer to the substrate.

12. The cartridge according to claim 8, wherein, for each well:
the bottom chamber is cylindrical with a first base radius; and
the top chamber is cylindrical with a second base radius greater than the first base radius,
wherein the bottom chamber and the top chamber extend along a same axis of symmetry.

13. The cartridge according to claim 8, wherein the first containment volume of the bottom chamber is comprised between 1.2 mm³ and 10.6 mm³, and the second containment volume of the top chamber is comprised between 3.8 mm³ and 19 mm³.

14. A manufacturing method of a chip for biological and/or biochemical analyses, comprising:
forming, in a supporting body including a substrate of semiconductor material, and a structural layer of biocompatible material extending over the substrate, a plurality of wells configured to contain a liquid solution for said biological and/or biochemical analyses, wherein forming the wells includes:
forming, at least in part in said structural layer, a bottom chamber defining a first containment volume for containing said liquid solution; and
forming, completely in said structural layer, a top chamber on top of, and in fluid communication with, the bottom chamber, and defining a second containment volume for containing the liquid solution, the second containment volume being greater than the first containment volume.

15. The method according to claim 14, wherein forming the bottom chamber and the top chamber comprise forming the bottom chamber and the top chamber completely in, and throughout a thickness of, the structural layer.

16. The method according to claim 14, wherein said structural layer is of a material having a refractive index lower than the refractive index of said liquid solution.

17. The method according to claim 16, wherein the liquid solution is water-based, the substrate is of silicon, and the structural layer is of polycarbonate.

18. The method according to claim 14, wherein the step of forming each well comprises:
etching selective portions of the substrate to form intermediate openings having a cylindrical shape with a first base radius;
forming, through the structural layer, a plurality of through openings having a cylindrical shape with the first base radius;
forming, in the structural layer, the top chambers having a cylindrical shape with a second base radius greater than the first base radius, in such a way that the through openings and the top chambers are in fluid communication with one another and aligned along a same axis of symmetry; and
positioning the structural layer on the substrate in such a way that the through openings, the top chambers, and the intermediate openings are aligned to one another along a same axis of symmetry.

19. The method according to claim 18, wherein positioning the structural layer on the substrate includes:
applying, in selective surface portions of the structural layer and/or of the substrate, a biocompatible adhesive layer; and
coupling the structural layer to the substrate with said biocompatible adhesive layer.

20. The method according to claim 14 wherein forming each well comprises:
forming, through the structural layer, a plurality of through openings having a cylindrical shape with a first base radius;
forming, in the structural layer, the top chambers having a cylindrical shape with a second base radius greater than the first base radius, in such a way that the through openings and the top chambers are in fluid communication with one another and aligned along a same axis of symmetry; and
positioning the structural layer in a flat region of the substrate in such a way that said flat region of the substrate forms a bottom wall for the through openings.
